(19) United States
(12) Patent Application Publication
(10) Pub. No.: US 2023/0044958 A1
(43) Pub. Date: Feb. 9, 2023

(54) METHOD OF TREATING VIRUS INFECTION USING A TLR7 AGONIST

(71) Applicant: Hoffmann-La Roche Inc., Little Falls, NJ (US)

(72) Inventors: Qingyan BO, Shanghai (CN); Yuyan JIN, Shanghai (CN); Gaurav TYAGI, Hillsdale, NJ (US); Yonghong ZHU, Shanghai (CN)

(73) Assignee: Hoffmann-La Roche Inc., Little Falls, NJ (US)

(21) Appl. No.: 17/788,487

(22) PCT Filed: Dec. 22, 2020
(86) PCT No.: PCT/EP2020/087561

§ 371 Date: Jun. 23, 2022

(30) Foreign Application Priority Data
Dec. 24, 2019 (CN) PCT/CN2019/127972
Aug. 28, 2020 (CN) PCT/CN2020/112137

(51) Int. Cl. A61K 31/429; A61K 31/522; A61P 31/20; A61P 31/14

(52) U.S. Cl. CPC A61K 31/429; A61K 31/522; A61P 31/20; A61P 31/14

(57) ABSTRACT

The present invention relates to methods of treating HBV, COVID-19 or SARS-CoV-2 infection in a human patient, wherein the methods comprise administration of a therapeutically effective amount of a TLR7 agonist, or a pharmaceutically acceptable salt thereof.

The present invention relates to method of treating HBV infection and/or viral hepatitis caused by HBV infection, or COVID-19 or SARS-CoV-2 infection in a human patient, wherein the method comprises administration of a therapeutically effective amount of TLR7 agonist, or a pharmaceutically acceptable salt thereof.

### FIELD OF THE INVENTION

Hepatitis B virus (HBV) infection is a major public health problem worldwide, roughly 30% of the world's population show serological evidence of current or past infection. Despite the introduction of a safe and effective prophylactic vaccine against the virus in the early 1980s, it is estimated that there are still more than 240 million chronic HBV carriers worldwide, a high percentage of whom will eventually develop liver cirrhosis or hepatocellular carcinoma (HCC) (WHO Hepatitis B. Fact Sheet No 204). In the 2010 Global Burden of Disease study (R Lozano, et al. Lancet, 380 (2012), 2095-2128), HBV infection ranked in the top health priorities in the world, and was the tenth leading cause of death (780,000 deaths per year). Recent studies have shown that progression to liver cirrhosis and HCC in patients with chronic HBV infection is significantly associated with circulating HBV DNA levels. Thus, antiviral therapy against HBV is critical to prevent the progression to cirrhosis or development of HCC.

Currently, there are two therapeutic classes available for the treatment of CHB: subcutaneously administered interferon (IFN) preparations (conventional or pegylated interferon-alpha [PEG IFN-α]) and orally administered nucleos(t)ide (NUCs; tenofovir (TFV) (including tenofovir disoproxil fumarate (TDF), an ester prodrug of tenofovir and tenofovir alafenamide (TAF), a phosphonate prodrug of TFV), entecavir (ETV), adefovir (ADV), telbivudine (LDT), clevudine (L-FMAU) and lamivudine (LAM or 3TC)). After 1 year of treatment, both types of treatment can suppress circulating HBV DNA levels (virologic response, 7%-94%), normalize serum liver transaminase enzymes (biochemical response, 32%-83%), and induce HBeAg seroconversion in HBeAg-positive patients (serological response, 10%-32%). Although these treatments reduce the risk of CHB sequelae, they are associated with very low rates of functional cure (HBsAg loss rates generally not exceeding 3% after one year of therapy) (EASL 2017). Furthermore, the existing standard-of-care therapies have important limitations. For example, virologic relapse after treatment discontinuation is a major limitation of currently approved therapies, which rarely result in functional cure. IFN based therapies have common adverse events of flu-like symptoms and can be associated with treatment-limiting adverse effects (e.g., neutropenia, thrombocytopenia), whilst NUCs require long-term and possibly lifelong therapy in the majority of treated patients. Given these limitations, there is an unmet need for novel treatments of a finite duration that yield higher functional cure rates (Liu et al 2017; Durantel and Zoulim 2016; Lok et al 2017; Wang and Chen 2014).

Toll-like receptors (TLRs) detect a wide range of conserved pathogen-associated molecular patterns (PAMPs). They play an important role of sensing invading pathogens and subsequent initiation of innate immune responses. There are 10 known members of the TLR family in human, which are type I transmembrane proteins featuring an extracellular leucine-rich domain and a cytoplasmic tail that contains a conserved Toll/interleukin (IL)-1 receptor (TIR) domain. Within this family, TLR7 is predominately expressed on plasmacytoid cells, and also on B-cells. Altered responsiveness of immune cells might contribute to the reduced innate immune responses during chronic viral infections. Agonist-induced activation of TLR7 might therefore represent a novel approach for the treatment of chronic viral infections. (D. J Connolly and L. AJ O'Neill, Current Opinion in Pharmacology 2012, 12:510-518, P. A. Roethle et al, J. Med. Chem. 2013, 56, 7324-7333).

As the application of TLR7 agonism in curing CHB is being developed, maximizing the immune stimulatory effects of TLR agonists without causing safety concern in human, both as single therapeutic agent or in combination therapy setting, should be carefully evaluated. Currently there was no best vial option for TLR7 dosing regimen in treating CHB. For example, Vesatolimod (GS-9620), a TLR7 agonist was tested with weekly dosing in virally suppressed patients with chronic hepatitis B for 4, 8, 12 weeks and did not show HBsAg decline (Jassen H L A. J Hepatology 2018). Additionally, this compound with weekly dosing for 12-week was also tested in combination with tenofovir disoproxil fumarate in viremic chronic hepatitis B (CHB) patients not currently on oral antiviral treatment and did not demonstrate any meaningful change in HBsAg after treatment (Agarwal K. J Viral Hepat 2018) Apparently, the optimal dosing regimen of TLR7 agonist, with or without in combination with other anti-HBV drug(s), remains unclear. Given the fact that immune non-responsive status does not help CHB patient to clear virus or viral antigen, therefore, novel treatment approach for TLR7 agonist-containing regimen should be considered in an effort to maximize its immune modulatory effect while minimizing the tachyphylaxis or immune tolerance phenomenon.

Since first reported as a mysterious pneumonia broke out in Wuhan, China in December, 2019 (Phelan A L et al, JAMA 2020). Coronavirus Disease 2019 (COVID-19) by the infection of the severe acute respiratory syndrome coronavirus 2 (SARS-CoV-2) has resulted in a pandemic with more than 18 million cases and 700 thousand deaths worldwide by early August, 2020. The clinical phenotypes of COVID-19 varies from asymptomatic disease or mild symptoms of the upper respiratory tract in most cases to potentially lethal, severe pneumonia with acute respiratory distress syndrome (ARDS) in up to 5% of patients tested positive for the virus (Richardson S et al JAMA. 2020). At present, only remdesivir was conditionally approved for severe form of COVID-19 by Health Authorities under emergency use authorization and its effect appears to be mediocre. There is an urgent need for the development of prophylactic medications (e.g. vaccine, convalescent serum antibody) and therapeutic medicines. The pathogenesis of COVID-19 is not fully known yet. Nevertheless, endosomal TLR7, a member of the pattern recognition receptor family, has been implicated in sensing the viral infections such as SARS-CoV-2. SARS-CoV-2 belongs to the broad family of viruses known as coronaviruses. It is a positive-sense single-stranded RNA (+ssRNA) virus, with a single linear RNA segment. TLR7 recognizes viral single-stranded RNA (ssRNA), resulting in the activation of TLR7 downstream signalling cascades, trigger the secretion of cytokines, among which type I/III interferons (IFNs) are considered crucial for antiviral defense. Furthermore, TLR activation facilitates and instructs the development of adaptive immune responses by upregulating the expression levels of co-stimulatory molecules such as CD80 and CD86 on dendritic cells (DCs), allowing the DCs to more effectively activate T cells (Tsuji S et al, Infect Immun 2000 and Michelsen K S et al, J Biol Chem 2001). Additionally, specific cytokines released via TLR7 activation may instruct differentiation of T cells into Th1 or Th2 subsets, which guide the pattern of adaptive response against the pathogen. Taken together, TLR7 programs antiviral defence in target cells and potentiates the adaptive immune response. If present early and properly localized, type I IFN can effectively limit CoV infection (Channappanavar R et al, Cell Host Microbe 2016, J Clin Invest. 2019). In July 2020, a study has demonstrated that a deficient TLR7 gene caused 4 young male COVID-19 patients to become seriously ill after being infected by SARS-CoV-2 (van der Made C I et al. JAMA. 2020). This suggests that TLR7 plays a key role in triggering the immune response for patients of COVID-19.

### SUMMARY OF THE INVENTION

In this invention, methods of the entire treatment comprising Compound (I) monotherapy or Compound (I) in combination with other anti-HBV drug(s) for 24-120 weeks were developed. Compound (I) is safe and well tolerated in chronic hepatitis B patients. Comparing with current NUC treatment, the current finite treatment methods of this invention not only minimize the risk of drug resistance and decrease adverse effects of NUC treatment (e.g. the risk of renal injury, bone density loss and creatine kinase elevation, etc), but also provide a better clinical outcome: the sustained HBV surface antigen (HBsAg) loss in a significantly larger portion among treated patients.

In this invention, the treatment methods demonstrate a robust HBV DNA and HBV RNA decline in patients. Furthermore, treatment method of Compound (I) in combination with other anti-HBV drugs results in significant hepatitis B surface antigen (HBsAg) decline or loss. In addition, currently there is no effective antiviral treatment for immune tolerant patients (defined as patients with highly replicative (HBV DNA levels elevated) and low inflammatory (ALT levels are normal or without signs of significant inflammation or fibrosis by liver biopsy)) and for patients with HBeAg-negative chronic HBV infection, previously termed as ‘inactive carrier’ characterized by the presence of serum antibodies to HBeAg (anti-HBe), undetectable or low (<2,000 IU/ml) HBV DNA levels and normal serum aminotransferases. However, the method of treatment of this invention with Compound (I) can also induce robust HBV DNA and HBV RNA decline in such patients.

Furthermore, in this invention, carefully designed treatment with off-period between on-periods (e.g. “On-Off-On”) maximizes the immune modulatory effect of the treatment period while leaving enough fine-tuning period that allows patients to be re-sensitized by subsequent course of the TLR7 agonist treatment to avoid tachyphylaxis or immune fatigue that is triggered by repetitive stimulation of an immune modulator like TLR7 agonist.

Additionally, methods of the entire treatment comprising Compound (I) monotherapy or Compound (I) in combination with other anti-COVID-19 or anti-SARS-CoV-2 drug(s) for 1-12 weeks were developed.

Finally, methods of dosing modification (titrate up or titrate down) of the adaptive treatment (multiple dose strengths and/or dosage regimens during a course of treatment) comprising Compound (I) monotherapy or Compound (I) in combination with other anti-HBV drug(s) for 24-120 weeks or anti-COVID-19/anti-SARS-CoV-2 drug(s) for 1-12 weeks were developed.

### BRIEF DESCRIPTION OF THE FIGURES

FIG. 1-A refers to regimens under conditions that TLR7 agonist Compound (I) is used in combination with other anti-HBV treatment which starts earlier than Compound (I) treatment. This is called “On-Off-On” theme that guarantees at least 2 cycles of Compound (I) treatment applied. The full course of “On-Off-On” treatment regimen is 24-96 weeks (exclusion of 0-24 week “Preparation-period” in FIG. 1-A). When a patient is eligible to receive treatment, it starts with the “Preparation-period”, defined as an optional treatment period when other anti-HBV drug treatment may be applied in advance. This period is 0-24 weeks. “Zero” week of “Preparation-period” indicates no other anti-HBV drug treatment is applicable up front “On-period”. The “On-period” is 6-24 weeks. After the completion of the 1^{st} “On-period”, the first “Off period” begins, which lasts 12-48 weeks with only other anti-HBV drug treatment in the absence of Compound (I). After the 1^{st} “Off period”, another “On period” of 6-24 weeks with treatment of Compound (I) upon other anti-HBV drug treatment can be repeated. After the 2^{nd} “On period”, repetitive cycles of the “Off period” followed by the “On period” can be applied. Alternatively, after the 2^{nd} “On period”, all treatment (including other anti-HBV drug treatment) can be stopped (as “option 1”) or other anti-HBV drug treatment is exclusively carried on until the end of entire treatment process (as “option 2”).

FIG. 1-B refers to regimen that TLR7 agonist Compound (I) is used as monotherapy, in the absence of other anti-HBV drug treatment. The entire treatment period is 24-108 weeks. The 1^{st} “On period” is the treatment period of Compound (I) in 6-24 weeks. The first “Off period” refers to period that is anti-HBV drug-free and lasts 12-48 weeks after the first “On period”. The next “On period” refers to the repetitive cycle of Compound (I) treatment, which is 6-24 weeks. After the 2^{nd} “On period” cycle, patient can be given additional alternate cycles of “Off period” and “On period” until the end of entire treatment process.

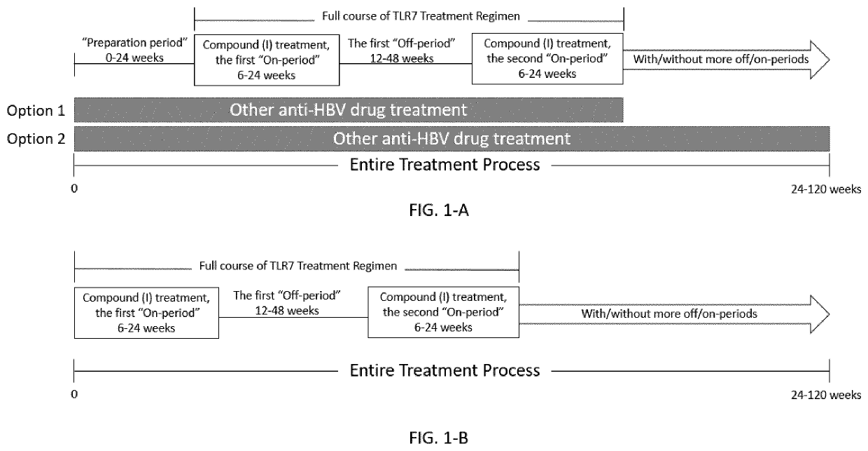

### DETAILED DESCRIPTION OF THE INVENTION

### Definitions

The term “NUC” denotes nucleos(t)ide analogs used as HBV therapy, including, but not limited to, lamivudine, adefovir dipivoxil, entecavir (ETV), telbivudine, clevudine, tenofovir disoproxil fumarate (TDF) and tenofovir alafenamide (TAF).

The term “immune checkpoint inhibitor” denotes antibodies, macrocyclic peptides or small molecules targeting PD-1/PD-L1, CTLA4 or VISTA pathways. Immune checkpoint inhibitor includes, but not limited to, nivolumab (BMS), pembrolizumab(Merck), aterolizumab (Roche/Genentech), avelumab (Merck KGaA/Pfizer), durvalumab (AstraZeneca), ipilimumab (BMS), tremelimumab (AstraZeneca), toripalimab (or JS001, Junshi Biosciences), Sintilimab (or IBI308, Innovent Biologics), camrelizumab (or SHR-1210, Henrui Medicine), tislelizumab (or BGB-A317, Beigene), ASC22 (or KN035, Alphamab/3D Medicines/Ascletis Pharma), HLX10 (Shanghai Henlius Biotech Inc) and CA-170 (Curis).

The term “CpAM” denotes specifically Class I compounds of HBV core protein allosteric modulators that induce aberrant capsids subsequently degraded, including, but not limited to, GLS4 (Sunshine Pharma), QL-007 (Qilu), KL060332 (Sichuan Kelun Pharmaceutical) and Compound (II) which was disclosed in WO2015132276.

The term “HBsAg loss” denotes quantitative HBsAg lower than limit of quantification (<0.05IU/mL) measured by Elecsys® HBsAg II (Roche) or other alternative quantitative HBsAg assays, such as Architect HBsAg QT assay by Abbott laboratories (Abbott, Chicago, Ill., USA), ETI-MAK-4 assay by DiaSorin (DiaSorin, Turin, Italy), Monolisa HBs Ag ULTRA assay by Bio-Rad (Bio-Rad Laboratories, Redmond, Wash., USA).

The term “IFN-α” denotes conventional interferon-α or pegylated interferon-α (PEG-IFN-α). Examples of IFN-α include, but not limited to, Pegasys® (Roche), PEG-Intron® (Merck& Co., Inc.) and Y-pegylated recombinant interferon alpha-2a (YPEG-IFNα-2a, Xiamen Amoytop Biotech Co., Ltd).

The term “retinoic acid-inducible Gene I” or “RIG-I” denotes a cytosolic pathogen recognition receptor that initiates the immune response against many RNA viruses. RIG-I modulator denotes a molecular which can induce type III and type I interferons through retinoic acid-inducible gene-I (RIG-I)-mediated sensing of the 5′-ε region of HBV pregenomic RNA. Example of RIG-I modulator includes, but not limited to, Inarigivir (SB9200) from Spring Bank Pharmaceuticals. (Sato S, Li K, Kameyama T, et al. The RNA sensor RIG-I dually functions as an innate sensor and direct antiviral factor for hepatitis B virus. Immunity 2015; 42: 123-32).

The term “Sting agonist” denotes agonists of stimulator of interferon genes (STING), a receptor that triggers an immune response when stimulated by pathogen DNA. Examples of Sting agonist include, but not limited to MK-1454 (Merck& Co., Inc.) and ADU-S100 (MIW815) (Aduro Biotech/Novartis).

The term “small interfering RNA” or “siRNA” denotes small interfering ribonucleic acid, which is a class of double-stranded RNA molecules. siRNA targets any viral transcript and induce its degradation by the RISC/Ago2 complex, resulting in gene silencing. Examples of siRNA includes, but not limited to, ARB-1467 (Arbutus Biopharma), ARO-HBV (Arrowhead Pharma), AB-729 (Arbutus Biopharma), DCR-HBVS (Dicerna), Vir-2218 (Alnylam and Vir Biotech), BB-103 (Benitec) and Lunar-HBV (Arcturus, USA with Janssen).

The term “HBV LNA” denotes a molecule which is a N-Acetylgalactosamine (GalNAc)-targeted locked nucleic acid (LNA)-containing single stranded oligodeoxyribonucleotide, complementary to hepatitis B virus (HBV) genome-derived mRNA species, intended for the treatment of CHB infections. Examples of HBV LNA includes, but not limited to, compound (III) which is any one of the compounds disclosed in WO2015/173208 or WO2014/179627.

The tem “HBV capsid inhibitor” denotes specifically Class II compounds of HBV core protein allosteric modulators that induce empty capsids with normal geometry and size. Examples of HBV capsid inhibitors are, but not limited to, ABI-H0731 (Assembly Biosciences), ABI-H2158 (Assembly Biosciences), AB-506 (Arbutus Biopharma), JNJ-6379 (Janssen), JNJ-0440 (Janssen).

The tem “HBV entry inhibitor” denotes compounds or Abs that disrupt the interaction between Na⁺-taurocholate co-transporting polypeptide (NTCP) (HBV entry receptor) and HBV particle/HBV surface proteins. Examples of HBV entry inhibitor includes, but not limited to, Myrcludex B (MYR Pharma).

The term “immune modulator/activator” refers to molecule that can upregulate innate and/or adaptive immunity of human through pathways of TLR7, TLR8, RIG-I, etc.

The term “HBV therapeutic vaccine” or “HBV vaccine” denotes a molecule which can stimulate or boost the host immune response to restore immune control, resulting in sustained suppression of HBV replication and ultimately HBsAg loss. Examples of HBV vaccine include, but not limited to, ABX203 (Center for Genetic Engineering and Biotechnology), INO-1800 (Inovio), HB-110 (Ichor Medical Systems with Janssen), TG1050 (Transgene) and HepTcell (Altimmune).

The term “HBsAg inhibitor” denotes molecules which can interfere with the assembly/release, production or entry of HBsAg. Examples of HBsAg inhibitor include, but not limited to, REP 2139 (Replicor), REP 2165 (Replicor).

The term “DAA” denotes “directing acting antiviral” drugs, which can be any direct acting anti-HBV agents such as HBV capsid inhibitor, HBV CpAM, HBV entry inhibitor (e.g. Myrcludex B), small interfering RNA (siRNA) targeting HBV transcripts at RNA level, HBsAg inhibitor (e.g. REP 2139 (Replicor) and REP 2165 (Replicor)).

The term “cccDNA destabilizer” denotes compounds that disrupt the presence of cccDNA in the infected liver cells. cccDNA destabilizers include, but not limited to, AB-452 (Arbutus).

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “anti-COVID-19” or “anti-SARS-CoV-2” drugs denote medicines used for the treatment and/or prophylactic use in suspicious/confirmed COVID-19 condition or SARS-CoV-2 infection, which can be any direct acting anti-SARS-CoV-2 agents, include but not limit to remdesivir (Gilead), REGN-COV2 (Regeneron), LY-CoV555 (Lilly), MK-4482/EIDD-2801 (Merck/Ridgeback Bio), or immune modulators, include but not limit to CD24Fc (Oncoimmune), T-COVID™(Altimmune), Itolizumab (Equillium Inc), or vaccine, include but not limit to AdCOVID™ (Altimmune), BNT162b1/2 (Pfizer/BioNTech), mRNA-1273 (Moderna), AZD1222/ChAdOx1 (AstraZeneca/Oxford Univ), Ad5-vectored COVID-19 vaccine (CanSino Biologics), CoronaVac (Sinovac), NVX-CoV2373 (Novavax).

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Method of Treatment

This invention is related to (i) method of treating HBV infection in a human patient, comprising administering to said patient a pharmaceutical composition containing an active ingredient of compound (I) in an amount from 50 mg to 200 mg QOD or QW or Q2W with or without other anti-HBV drugs; wherein compound (I) is (1S)-1-[(2S,4R,5R)-5-(5-amino-2-oxo-thiazolo[4,5-d]pyrimidin-3-yl)-4-hydroxy-tetrahydrofuran-2-yl]propyl] acetate or pharmaceutically acceptable salt; wherein the entire treatment process comprises two or three Compound (I) treatment periods (“on-period”), and each adjacent on-periods are separated by one “off-period” during which Compound (I) is not treated.

A further embodiment of present invention is (ii) the method according to (i), wherein the entire treatment process starts with a preparation period; wherein the preparation period is 0-24 weeks; particularly 0 week, 4 weeks, 8 weeks, 12 weeks, 16 weeks, 20 weeks or 24 weeks.

A further embodiment of present invention is (iii) the method according to (i) or (ii), wherein the on-period is 6-24 weeks; off-period is 12-48 weeks; and the entire treatment process is 24-120 weeks, particularly 24 weeks, 36 weeks, 48 weeks, 60 weeks, 72 weeks, 84 weeks, 96 weeks, 108 weeks or 120 weeks.

A further embodiment of present invention is (iv) the method according to any one of (i) to (iii), wherein compound (I) is administered at dose of 50 mg QOD, 100 mg QOD, 150 mg QOD, or 200 mg QOD without other anti-HBV drugs; wherein the entire treatment process is 48 weeks, preparation period is 0 week, the first on-period is 8-16 weeks, the first off-period is 16-32 weeks, the second on-period is 8-16 weeks.

A further embodiment of present invention is (v) the method according to any one of (i) to (iv), wherein compound (I) is administered at dose of 50 mg QOD, 100 mg QOD, 150 mg QOD, or 200 mg QOD without other anti-HBV drugs; wherein the entire treatment process is 36 weeks, preparation period is 0 week, the first on-period is 12 weeks, the first off-period is 12 weeks, the second on-period is 12 weeks.

A further embodiment of present invention is (vi) the method according to any one of (i) to (v), wherein compound (I) is administered at dose of 50 mg QOD, 100 mg QOD, 150 mg QOD, or 200 mg QOD with other anti-HBV drugs, the other anti-HBV drugs are administered to the patient on the first day of the entire treatment process.

A further embodiment of present invention is (vii) the method according to any one of (i) to (vi), wherein the other anti-HBV drugs are one or two agents independently selected from anti-HBV nucleos(t)ide analogues, immune checkpoint inhibitor, immune activator, HBV CpAM, HBV capsid inhibitor, RIG-I agonist, Sting agonist, HBV therapeutic vaccine, HBV LNA, HBV entry inhibitor, cccDNA destabilizers, siRNA and HBsAg inhibitor.

A further embodiment of present invention is (viii) the method according to any one of (i) to (vi), wherein the other anti-HBV drugs are one or two agents independently selected from ETV, TDF, TAF, lamivudine, telbivudine, clevudine, nivolumab, pembrolizumab, aterolizumab, avelumab, durvalumab, ipilimumab, tremelimumab, toripalimab, Sintilimab, camrelizumab, tislelizumab, ASC22, HLX10, CA-170, GLS4, QL-007, KL060332, compound (II), ABI-H0731, ABI-H2158, AB-506, JNJ-6379, JNJ-0440, Inarigivir, MK-1454, ADU-S100, ABX203, INO-1800, HB-110, TG1050, HepTcell, compound (III), Myrcludex B, AB-452, ARB-1467, ARO-HBV, AB-729, DCR-HBVS, Vir-2218, BB-103, Lunar-HBV, REP 2139 and REP 2165.

A further embodiment of present invention is (ix) the method according to any one of (i) to (vii), wherein the other anti-HBV drug is HBV CpAM.

A further embodiment of present invention is (x) the method according to any one of (i) to (ix), wherein the HBV CpAM is compound (II), 3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-15 2,2-dimethyl-propanoic acid, which is dosed at 200-1000 mg QD, particularly at 200 mg QD, 400 mg QD, 600 mg QD, 800 mg QD or 1000 mg QD.

A further embodiment of present invention is (xi) the method according to any one of (i) to (x), wherein the entire treatment process is 48 weeks, the preparation period is 12 weeks, the first on-period is 12 weeks, the first off-period is 12 weeks, the second on-period is 12 weeks.

A further embodiment of present invention is (xii) the method according to any one of (i) to (xi), wherein the entire treatment process is 48 weeks, the preparation period is 0-8 weeks, the first on-period is 8-16 weeks, the first off-period is 12-24 weeks, the second on-period is 8-16 weeks.

A further embodiment of present invention is (xiii) the method according to any one of (i) to (xii), wherein the other anti-HBV drugs are HBV CpAM and NUC.

A further embodiment of present invention is (xiv) the method according to any one of (i) to (xiii), wherein the HBV CpAM is compound (II) which is dosed at 200-1000 mg QD, particularly at 200 mg QD, 400 mg QD, 600 mg QD, 800 mg QD or 1000 mg QD; wherein NUC is ETV, TDF or TAF, which is dosed according to its country-specific label.

A further embodiment of present invention is (xv) the method according to any one of (i) to (xiv), wherein the entire treatment process is 48 weeks, the preparation period is 12 weeks, the first on-period is 12 weeks, the first off-period is 12 weeks, the second on-period is 12 weeks.

A further embodiment of present invention is (xvi) the method according to any one of (i) to (xv), wherein the entire treatment process is 48 weeks, the preparation period is 0-8 weeks, the first on-period is 8-16 weeks, the first off-period is 12-24 weeks, the second on-period is 8-16 weeks.

A further embodiment of present invention is (xvii) the method according to any one of (i) to (xvi), wherein compound (II) is administered at dose of 600 mg QD.

A further embodiment of present invention is (xviii) the method according to any one of (i) to (xvii), wherein the other anti-HBV drug is immune checkpoint inhibitor.

A further embodiment of present invention is (xix) the method according to any one of (i) to (xviii), wherein the immune checkpoint inhibitor is antibody, macrocyclic peptide or small molecule targeting PD-1/PD-L1, CTLA4 or VISTA pathways.

A further embodiment of present invention is (xx) the method according to any one of (i) to (xix), wherein the immune checkpoint inhibitor is selected from nivolumab, pembrolizumab, aterolizumab, avelumab, durvalumab, ipilimumab, tremelimumab, toripalimab, Sintilimab, camrelizumab, tislelizumab, ASC22, HLX10, and CA-170; particularly the immune check point inhibitor is nivolumab.

A further embodiment of present invention is (xxi) the method according to any one of (i) to (xx), wherein the entire treatment process is 48 weeks, the preparation period is 12 week, the first on-period is 12 weeks, the first off-period is 12 weeks, the second on-period is 12 weeks.

A further embodiment of present invention is (xxii) the method according to any one of (i) to (xxi), wherein the entire treatment process is 36 weeks, the preparation period is 0 week, the first on-period is 12 weeks, the first off-period is 12 weeks, the second on-period is 12 weeks.

A further embodiment of present invention is (xxiii) the method according to any one of (i) to (xxii), wherein the nivolumab is administered at dose of 0.3-5 mg/kg Q2W, Q3W or Q4W.

A further embodiment of present invention is (xxiv) the method according to any one of (i) to (xxiii), wherein compound (I) is administered at dose of 150 mg QOD.

A further embodiment of present invention is (xxv) the method according to any one of (i) to (xxiv), wherein compound (I) is administered at dose of 100 mg QOD.

A further embodiment of present invention is (xxvi) the method according to any one of (i) to (xxv), wherein compound (I) is administered at high doses of 150 or 200 mg QOD, which is switched to low doses of 50 mg QOD or 100 mg QOD and then still has the option of switching to 50 mg QW or 100 QW, wherein the dose change happens at any time during the treatment period.

A further embodiment of present invention is (xxvii) the method according to any one of (i) to (xxvi), wherein compound (I) is administered at low doses of 50 or 100 mg QW, which is switched to 50 mg QOD or 100 mg QOD and then still has the option of switching to 150 mg QOD or 200 mg QOD; or initially compound (I) is administered at low doses of 50 or 100 mg QOD but is switched to 150 mg QOD or 200 mg QOD; wherein the dose change happens at any time during the treatment period.

Another embodiment of present invention is (xxviii) a method of treating COVID-19 or SARS-CoV-2 infection in a human patient or treating subject for SARS-CoV-2 or COVID-19 prophylaxis, comprising administering to said patient or subject a pharmaceutical composition containing an active ingredient of compound (I) in an amount from 50 mg to 200 mg QOD or QW or Q2W with or without one or more other anti-COVID-19 or anti-SARS-CoV-2 drugs; wherein compound (I) is (1S)-1-[(2S,4R,5R)-5-(5-amino-2-oxo-thiazolo[4,5-d]pyrimidin-3-yl)-4-hydroxy-tetrahydrofuran-2-yl]propyl] acetate or pharmaceutically acceptable salt; wherein Compound (I) is used in the treatment period of 1-12 weeks.

A further embodiment of present invention is (xxix) the method according to (xxviii), wherein compound (I) is administrated at 50 mg QOD or QW or Q2W, 100 mg QOD or QW or Q2W, 150 mg QOD or QW or Q2W, or 200 mg QOD or QW or Q2W.

A further embodiment of present invention is (xxx) the method according to any one of (xxviii) to (xxix), wherein the anti-COVID-19 or anti-SARS-CoV-2 drug is independently selected from direct acting anti-SARS-CoV-2 agents, immune modulators and vaccine.

A further embodiment of present invention is (xxxi) the method according to any one of (xxviii) to (xxx), wherein the anti-COVID-19 or anti-SARS-CoV-2 drug is independently selected from remdesivir, REGN-COV2, LY-CoV555, MK-4482/EIDD-2801, CD24Fc, T-COVID™, Itolizumab, AdCOVID™, BNT162b1/2, mRNA-1273, AZD1222/ChAdOx1, Ad5-vectored COVID-19 vaccine, CoronaVac, and NVX-CoV2373.

A further embodiment of present invention is (xxxii) the method according to any one of (xxviii) to (xxxi), wherein compound (I) is administered at high doses of 150 or 200 mg QOD, which is switched to low doses of 50 mg QOD or 100 mg QOD and then still has the option of switching to 50 mg QW or 100 QW, wherein the dose change happens at any time during the treatment period.

A further embodiment of present invention is (xxxiii) the method according to any one of (xxviii) to (xxxii), wherein compound (I) is administered at low doses of 50 or 100 mg QW, which is switched to 50 mg QOD or 100 mg QOD and then still has the option of switching to 150 mg QOD or 200 mg QOD; or initially compound (I) is administered at low doses of 50 or 100 mg QOD but is switched to 150 mg QOD or 200 mg QOD; wherein the dose change happens at any time during the treatment period.

A further embodiment of present invention is (xxxiv) the method according to any one of (i) to (xxxiii), wherein compound (I) is administered at doses of 50 to 200 mg, twice weekly with dosing interval between 1-4 days (e.g. Monday/Wednesday dosing, Monday/Thursday dosing, Monday/Friday dosing, Monday/Saturday dosing, Tuesday/Thursday dosing, Tuesday/Friday dosing, Tuesday/Saturday dosing, Tuesday/Sunday dosing), or three times weekly with dosing interval between 1-2 days (e.g. Monday/Wednesday/Friday, Monday/Wednesday/Saturday, Monday/Thursday/Saturday, Tuesday/Thursday/Saturday, Tuesday/Thursday/Sunday, Tuesday/Friday/Sunday). The dose can also start from 50 mg or 100 mg QOD at first (twice/three times weekly) and then has the option of switching to 150 mg or 200 mg (twice/three times weekly); or initially compound (I) is administered at high doses of 150 or 200 mg at first (twice/three times weekly) but is switched to 50 mg or 100 mg later (twice/three times weekly); wherein the dose change happens at any time during the treatment period.

### Indications and Methods of Treatment

Another embodiment provides pharmaceutical compositions or medicaments containing the compounds of the invention and a therapeutically inert carrier, diluent or excipient, as well as methods of using the compounds of the invention to prepare such compositions and medicaments. In one example, Compound (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a Compound (I) is formulated in an acetate buffer, at pH 5. In another embodiment, the Compound (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to be potent as a CpAM, which can be used, but not limited, for the treatment or prevention of hepatitis B virus infected patients.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

### EXAMPLES

The invention will be more fully understood by reference to the following examples. They should not, however, be construed as limiting the scope of the invention.

### Abbreviations

Abbreviations used herein are as follows:
AASLD: American Association for the Study of the Liver
APASL: Asia-Pacific Association for the Study of the Liver
BID: twice a day
CHB: chronic hepatitis B
DAA: direct-acting antiviral
EASL: European Association for the Study of the Liver
ETV: entecavir
Ex.: example
NUC: nucleos(t)ide analog
HBeAg: hepatitis B e antigen
HBsAg: hepatitis B surface antigen
HBV: hepatitis B virus
HCC: hepatocellular carcinoma
HLM human liver microsome
PEG-IFN: pegylated interferon
QD: once a day
QOD: every other day
QW: once a week
Q2W: once every two weeks
Q3W: once every three weeks
Q4W: once every four weeks
TAF: tenofovir alafenamide.
TDF: tenofovir disoproxil fumarate
WHO: World Health Organization

### MATERIALS AND METHODS

### Compounds:

Compound (I): a TLR7 agonist with the following structure, which was disclosed in WO2016091698.

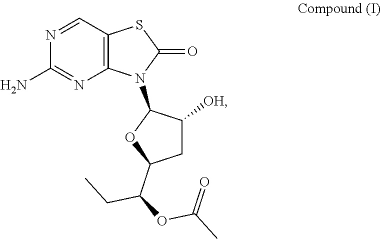 [SMILES: CC[C@H](OC(C)=O)[C@@H]1C[C@@H](O)[C@H](n2c(=O)sc3cnc(N)nc32)O1]

(1S)-1-[(2S,4R,5R)-5-(5-amino-2-oxo-thiazolo[4,5-d]pyrimidin-3-yl)-4-hydroxy-tetrahydrofuran-2-yl]propyl] acetate. Compound (II): an HBV CpAM with the following structure, which was disclosed in WO2015132276.

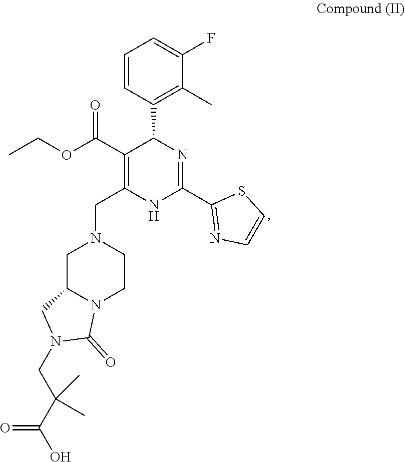 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]

3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-15 2,2-dimethyl-propanoic acid. Compound (III): an HBV LNA oligonucleotide, which is any one of the compounds disclosed in WO2015/173208 or WO2014/179627.

### Study Subjects:

Patients who have been confirmed or suspicious of chronic HBV infection or chronic hepatitis B as judged by healthcare professional or lab test with any one or more of the following conditions:
1) HBsAg positive and/or quantifiable;
2) HBV DNA detectable and/or quantifiable by an HBV DNA measurement;
3) HBeAg positive;
4) Anti-HBcAg positive;
5) Diagnosed as one of the following diseases: HBeAg-positive chronic HBV infection (Immune tolerant), HBeAg-positive CHB (Immune reactive), HBeAg-negative CHB, HBeAg-negative chronic HBV infection (Inactive carrier) by either one of the following treatment guidelines: AASLD Guidelines for Treatment of Chronic Hepatitis B (2018), EASL HBV Guideline 2017, APASL HBV Guidelines 2015, and any other applicable guidelines that are country-specific and/or applicable to the physicians.

### General Method

After been tested as positive of HBV infection and suitable for clinical care by physician, some patients will be initially and continuously given a standard antiviral treatment regimen including but not limited to, nucleos(t)ide analogue treatment. These patients are categorized as NUC-suppressed and/or on-treatment patients in general (including treatment other than NUC). Another group of patients who have never been treated or have stopped treatment after initial treatment (still have ongoing HBV infection) are categorized as treatment-naive and/or not-on-treatment patients in general.

In one embodiment, the on-treatment CHB patients (e.g. NUC-suppressed patients) were treated initially with other anti-HBV drug(s), once the physician decides to add Compound (I) (TLR7 agonist) on top of the existing treatment, a “preparation period” of 0-24 weeks is created before dosing the Compound (I) (TLR7 agonist) while the patients are still under the original treatment. When Compound (I) (TLR7 agonist) treatment starts, either one, two or three Compound (I) (TLR7 agonist) treatment periods (hereinafter as “on-period”) that are 6-24 weeks can be applied. If two or three on-periods are to be applied, it is mandatory that each “on-period” should be separated by one TLR7 pharmaceutical composition-free interval (hereinafter as “off-period”) which is 12-48 weeks. Nevertheless, the original existing treatment (e.g. NUC) should be still applied during the “off-period”.

In another embodiment, for the treatment-naive and/or not-on-treatment patients, once the physician decides to start Compound (I) (TLR7 agonist) containing regimen, either one, two or three on-periods that are 6-24 weeks can be applied. The Compound (I) (TLR7 agonist) containing regimen refers to either Compound (I) (TLR7 agonist) monotherapy or Compound (I) (TLR7 agonist) in combination with other anti-HBV drug(s). If two or three On-periods are to be applied, it is mandatory that each “on-period” should be separated by one “off-period” which is 12-48 weeks. Nevertheless, if a Compound (I) (TLR7 agonist) combination therapy regimen is used (Compound (I) plus other anti-HBV drug(s)), the other anti-HBV drug(s) should be still applied during the “off-period”.

In another embodiment of “double whammy” immune stimulation regimen, for the treatment-naive and/or not-on-treatment patients, once the physician decides to add a TLR7 agonist-containing regimen upon original treatment with non-TLR7 agonist immune checkpoint inhibitor, either one, two or three or more on-periods that are 6-24 weeks can be applied. The double immune stimulation regimen refers to TLR7 agonist in combination with another immune checkpoint inhibitor, including but not limit to, anti-PD1 or anti-PD-L1 antibody (e.g. nivolumab, ASC22). If two or three or more TLR7 agonist treatment periods/cycles are to be applied, it is mandatory that each “On-period” should be separated by one off-period which is 12-24 weeks. Nevertheless, the original non-TLR7 agonist immune checkpoint inhibitor should be still applied during the off-period.

Treatments with different “off-period” or “on-period” can be called as, for example, “On-Off-On”, “On-Off-On-Off”, “On-Off-On-Off-On”, “Off-On”, “Off-On-Off-On”, “Off-On-Off-On-Off-On” approaches, and so on. An off-period is essential to maximize the immune modulatory effect of the treatment period prior to the Off-period while leaving enough fine-tuning period that allows patients to be re-sensitized by subsequent course of the TLR7 agonist treatment to avoid tachyphylaxis or immune fatigue that is triggered by repetitive stimulation of an immune modulator like TLR7 agonist.

The entire process of treatment is at least 24 weeks, particularly 24 weeks, 48 weeks, 60 weeks, 72 weeks, 84 weeks, 96 weeks, 108 weeks or 120 weeks.

The approaches mentioned above can be applied as the initial anti-HBV treatment to the patients, or serve as the additional treatment after any other anti-HBV treatment, or simply added on top of any ongoing anti-HBV treatment to the patients.

In another embodiment, the other anti-HBV drug(s) used in the entire process of treatment approaches mentioned above include, but not limited to, anti-HBV nucleos(t)ide analogues, immune checkpoint inhibitor (e.g. anti-PD-1, anti-PD-L1, anti-CTLA4), immune activator (e.g. RIG-I agonist, Sting agonist, HBV therapeutic vaccine), HBV capsid inhibitor, HBV CpAM (e.g. GLS4, QL-007, KL060332 and compound (II)), HBV RNA knockdown agent (e.g. HBV LNA), HBV entry inhibitor, cccDNA destabilizers, small interfering RNA (siRNA), HBsAg inhibitor. In another embodiment, the treatment of other antiviral drugs can be continuous dosing or dosing according to its specific label, however the administration of TLR7 agonist at a dose of 50-200 mg QOD or QW or Q2W orally is applied in the treatment approaches mentioned above.

### Examples of Tests

The following examples are intended to illustrate the meaning of the present invention but should by no means represent a limitation within the meaning of the present invention:

### Example (I) Treatment for HBV Infection

The following examples follow the protocol described in general method above, specific parameters were disclosed in Table 1 as below:

TABLE 1
Examples of dose regimen

| | | Dose and | | | | | |
|---|---|---|---|---|---|---|---|
| | Other anti- | administration | Entire | | The first | The first | The second |
| | HBV drug(s) and | frequency of | treatment | Preparation | “On- | “Off- | “On- |
| Ex. | administration | Compound (I) | process | period | period” | period” | period” |
| 1 | Compound | 50, 100, 150 or | 48 | 12 | 12 | 12 | 12 |
| | (II): 200, 400, | 200 mg; QOD | weeks | weeks | weeks | weeks | weeks |
| | 600, 800 or | | | | | | |
| | 1000 mg; QD | | | | | | |
| 2 | Compound | 50, 100, 150 or | 48 | 12 | 12 | 12 | 12 |
| | (II): 200, 400, | 200 mg; QOD | weeks | weeks | weeks | weeks | weeks |
| | 600, 800 or | | | | | | |
| | 1000 mg QD; | | | | | | |
| | and NUC | | | | | | |
| | (ETV or TDF | | | | | | |
| | or TAF): per | | | | | | |
| | country- | | | | | | |
| | specific label | | | | | | |
| 3 | Compound | 150 mg; QOD | 48 | 12 | 12 | 12 | 12 |
| | (II): 600 mg; | | weeks | weeks | weeks | weeks | weeks |
| | QD | | | | | | |
| 4 | Compound | 150 mg; QOD | 48 | 12 | 12 | 12 | 12 |
| | (II): 600 mg | | weeks | weeks | weeks | weeks | weeks |
| | QD; and ETV: | | | | | | |
| | per country- | | | | | | |
| | specific label | | | | | | |
| 5 | Compound | 150 mg; QOD | 48 | 12 | 12 | 12 | 12 |
| | (II): 600 mg | | weeks | weeks | weeks | weeks | weeks |
| | QD; and TDF: | | | | | | |
| | per country- | | | | | | |
| | specific label | | | | | | |
| 6 | Compound | 150 mg; QOD | 48 | 12 | 12 | 12 | 12 |
| | (II): 600 mg | | weeks | weeks | weeks | weeks | weeks |
| | QD; and TAF: | | | | | | |
| | per country- | | | | | | |
| | specific label | | | | | | |
| 7 | DAA: per | 50, 100, 150 or | 48 | 12 | 12 | 12 | 12 |
| | country- | 200 mg; QOD | weeks | weeks | weeks | weeks | weeks |
| | specific label | | | | | | |
| 8 | DAA and | 50, 100, 150 or | 48 | 12 | 12 | 12 | 12 |
| | NUC: per | 200 mg; QOD | weeks | weeks | weeks | weeks | weeks |
| | country- | | | | | | |
| | specific label | | | | | | |
| 9 | Compound | 50, 100, 150 or | 48 | 0-8 | 8-16 | 12-24 | 8-16 |
| | (II): 200-1000 | 200 mg; QOD | weeks | weeks | weeks | weeks | weeks |
| | mg QD | | | | | | |
| 10 | Compound | 50, 100, 150 or | 48 | 0-8 | 8-16 | 12-24 | 8-16 |
| | (II): 200-1000 | 200 mg; QOD | weeks | weeks | weeks | weeks | weeks |
| | mg QD; and | | | | | | |
| | NUC (ETV or | | | | | | |
| | TDF or TAF): | | | | | | |
| | per country- | | | | | | |
| | specific label | | | | | | |
| 11 | DAA: per | 50, 100, 150 or | 48 | 0 | 8-16 | 16-32 | 8-16 |
| | country- | 200 mg; QOD | weeks | week | weeks | weeks | weeks |
| | specific label | | | | | | |
| 12 | DAA and | 50, 100, 150 or | 48 | 0 | 8-16 | 16-32 | 8-16 |
| | NUC: per | 200 mg; QOD | weeks | week | weeks | weeks | weeks |
| | country- | | | | | | |
| | specific label | | | | | | |
| 13 | None | 50, 100, 150 or | 48 | 0 | 8-16 | 16-32 | 8-16 |
| | | 200 mg; QOD | weeks | week | weeks | weeks | weeks |
| 14 | None | 50, 100, 150 or | 36 | 0 | 12 | 12 | 12 |
| | | 200 mg; QOD | weeks | weeks | weeks | weeks | weeks |
| 15 | None | 150 mg; QOD | 48 | 0 | 8-16 | 16-32 | 8-16 |
| | | | weeks | week | weeks | weeks | weeks |
| 16 | None | 150 mg; QOD | 36 | 0 | 12 | 12 | 12 |
| | | | weeks | weeks | weeks | weeks | weeks |
| 17 | Nivolumab: | 50, 100, 150 or | 48 | 12 | 12 | 12 | 12 |
| | 0.3-5 mg/kg; | 200 mg; QOD | weeks | weeks | weeks | weeks | weeks |
| | Q2W, Q3W or | | | | | | |
| | Q4W | | | | | | |
| 18 | Nivolumab: | 50, 100, 150 or | 36 | 0 | 12 | 12 | 12 |
| | 0.3-5 mg/kg; | 200 mg; QOD | weeks | weeks | weeks | weeks | weeks |
| | Q2W, Q3W or | | | | | | |
| | Q4W | | | | | | |
| 19 | Nivolumab: | 150 mg; QOD | 48 | 12 | 12 | 12 | 12 |
| | 0.3-5 mg/kg; | | weeks | weeks | weeks | weeks | weeks |
| | Q2W, Q3W or | | | | | | |
| | Q4W | | | | | | |
| 20 | Nivolumab: | 150 mg; QOD | 36 | 0 | 12 | 12 | 12 |
| | 0.3-5 mg/kg; | | weeks | weeks | weeks | weeks | weeks |
| | Q2W, Q3W or | | | | | | |
| | Q4W | | | | | | |
| 21 | DAA: per | 50, 100, 150 or | 48-120 | 24 | 6-24 | 12-48 | 6-24 |
| | country- | 200 mg; QOD | weeks | weeks | weeks | weeks | weeks |
| | specific label | | | | | | |
| 22 | DAA and | 50, 100, 150 or | 48-120 | 24 | 6-24 | 12-48 | 6-24 |
| | NUC: per | 200 mg; QOD | weeks | weeks | weeks | weeks | weeks |
| | country- | | | | | | |
| | specific label | | | | | | |
| 23 | Compound | 50, 100, 150 or | 24-120 | 0-24 | 6-24 | 12-48 | 6-24 |
| | (II): 200, 400, | 200 mg; QOD | weeks | weeks | weeks | weeks | weeks |
| | 600, 800 or | | | | | | |
| | 1000 mg QD | | | | | | |
| 24 | Compound | 50, 100, 150 or | 24-120 | 0-24 | 6-24 | 12-48 | 6-24 |
| | (II): 200, 400, | 200 mg; QOD | weeks | weeks | weeks | weeks | weeks |
| | 600, 800 or | | | | | | |
| | 1000 mg QD; | | | | | | |
| | and NUC | | | | | | |
| | (ETV or TDF | | | | | | |
| | or TAF): per | | | | | | |
| | country- | | | | | | |
| | specific label | | | | | | |

For patients in the treatment of combination therapy as list above, they can be either previously on initial treatment or naive when they start.

Furthermore, compound (I) can be administered at doses of 50 to 200 mg, twice weekly with dosing interval between 1-4 days (e.g. Monday/Wednesday dosing, Monday/Thursday dosing, Monday/Friday dosing, Monday/Saturday dosing, Tuesday/Thursday dosing, Tuesday/Friday dosing, Tuesday/Saturday dosing, Tuesday/Sunday dosing), or three times weekly with dosing interval between 1-2 days (e.g. Monday/Wednesday/Friday, Monday/Wednesday/Saturday, Monday/Thursday/Saturday, Tuesday/Thursday/Saturday, Tuesday/Thursday/Sunday, Tuesday/Friday/Sunday). The dose can also start from 50 mg or 100 mg QOD at first (twice/three times weekly) and then has the option of switching to 150 mg or 200 mg (twice/three times weekly); or initially compound (I) is administered at high doses of 150 or 200 mg at first (twice/three times weekly) but is switched to 50 mg or 100 mg later (twice/three times weekly); wherein the dose change happens at any time during the treatment period.

### Example (II) Treatment for Less Tolerable Patients for HBV Infection

Compound (I) is initially administered at high doses of 150 or 200 mg QOD, which is switched to low doses of 50 mg QOD or 100 mg QOD and then still has the option of switching to 50 mg QW or 100 QW based on pharmacodynamics responses (e.g. IP-10, cytopenia) and/or flu-like symptoms (e.g. fever, nausea, headache, myalgia) and/or tolerability of patients to compound (I). Dose switching can happen at any time during the treatment period.

Compound (I) is initially administered at low doses of 50 or 100 mg QW, which is switched to 50 mg QOD or 100 mg QOD and then still has the option of switching to 150 mg QOD or 200 mg QOD; or initially compound (I) is administered at low doses of 50 or 100 mg QOD but is switched to 150 mg QOD or 200 mg QOD based on pharmacodynamics responses (e.g. IP-10, cytopenia) and/or flu-like symptoms (e.g. fever, nausea, headache, myalgia) and/or tolerability of patients to compound (I). Dose switching can happen at any time during the treatment period.

### Example (III) Treatment for COVID-19 or SARS-CoV-2 Infection or Prophylaxis

After been tested or suspected as positive of SARS-CoV-2 infection or COVID-19 and suitable for compound (I) clinical care by physician, some patients will be initially and/or continuously given a standard anti-SARS-CoV-2 and/or anti-COVID-19 treatment regimen while some patients will not.

Once the physician decides to add Compound (I) (TLR7 agonist) on top of the existing treatment or as the only anti-SARS-CoV-2/anti-COVID-19 treatment, compound (I) (TLR7 agonist) will be given for one treatment period of 1-12 week.

The approaches mentioned above can be applied as the initial anti-SARS-CoV-2 or anti-COVID-19 treatment to the patients, or serve as the additional treatment after any other anti-SARS-CoV-2 or anti-COVID-19 treatment, or simply added on top of any ongoing treatment to the patients, or starting compound (I) treatment first and then simply add on additional anti-SARS-CoV-2 or anti-COVID-19 treatment.

Once the physician decides to use Compound (I) (TLR7 agonist) to treat patients, methods of dosing modification (titrate up or titrate down) of the adaptive treatment (multiple dose strengths and/or dosage regimens during a course of treatment) comprising Compound (I) monotherapy or Compound (I) in combination with other anti-COVID-19 or anti-SARS-CoV-2 drugs can be applied, wherein Compound (I) is initially administered at high doses of 150 or 200 mg QOD, but is switched to low doses of 50 mg QOD or 100 mg QOD and then still has the option of switching to 50 mg QW or 100 QW based on pharmacodynamics responses (e.g. IP-10, IFN alpha, cytopenia) and/or flu-like symptoms (e.g. fever, nausea, headache, myalgia) and/or tolerability of patients to Compound (I) and/or physician's assessment. Dose change can happen at any time during the treatment period.

Alternatively, Compound (I) can be initially administered at low doses of 50 or 100 mg QW but is switched to 50 mg QOD or 100 mg QOD and then can go to the option of switching to 150 mg QOD or 200 mg QOD; or initially administered at low doses of 50 or 100 mg QOD but is switched to 150 mg QOD or 200 mg QOD based on pharmacodynamics responses (e.g. IP-10, IFN alpha, cytopenia) and/or flu-like symptoms (e.g. fever, nausea, headache, myalgia) and/or tolerability of patients to Compound (I) and/or physician's assessment. Dose change can happen at any time during the treatment period.

When physician decides to give Compound (I) to subjects without SARS-CoV-2 or COVID-19 infection for prophylactic use, a full course of Compound (I) at 50 mg QOD or QW, 100 mg QOD or QW, 150 mg QOD or QW, or 200 mg QOD or QW for 1-12 weeks can be taken for preventive purpose, with or without the use of COVID-19/SARS-CoV-2 vaccine or convalescent serum.

Once the physician decides to use Compound (I) (TLR7 agonist) to treat subjects without COVID-19/SARS-CoV-2 infection, methods of dosing modification (titrate up or titrate down) of the adaptive treatment (multiple dose strengths and/or dosage regimens during a course of treatment) comprising Compound (I) monotherapy or Compound (I) in combination with vaccine or convalescent serum can be applied, wherein Compound (I) is initially administered at high doses of 150 or 200 mg QOD but is switched to low doses of 50 mg QOD or 100 mg QOD and then still has the option of switching to 50 mg QW or 100 QW based on pharmacodynamics responses (e.g. IP-10, IFN alpha, cytopenia) and/or flu-like symptoms (e.g. fever, nausea, headache, myalgia) and/or tolerability of patients to compound (I) and/or physician's assessment.

Alternatively, Compound (I) can be initially administered at low doses of 50 or 100 mg QW but is switched to 50 mg QOD or 100 mg QOD and then can go to the option of switching to 150 mg QOD or 200 mg QOD; or initially administered at low doses of 50 or 100 mg QOD but is switched to 150 mg QOD or 200 mg QOD based on pharmacodynamics responses (e.g. IP-10, IFN alpha, cytopenia) and/or flu-like symptoms (e.g. fever, nausea, headache, myalgia) and/or tolerability of subjects to Compound (I) and/or physician's assessment.

Furthermore, compound (I) can be administered at doses of 50 to 200 mg, twice weekly with dosing interval between 1-4 days (e.g. Monday/Wednesday dosing, Monday/Thursday dosing, Monday/Friday dosing, Monday/Saturday dosing, Tuesday/Thursday dosing, Tuesday/Friday dosing, Tuesday/Saturday dosing, Tuesday/Sunday dosing), or three times weekly with dosing interval between 1-2 days (e.g. Monday/Wednesday/Friday, Monday/Wednesday/Saturday, Monday/Thursday/Saturday, Tuesday/Thursday/Saturday, Tuesday/Thursday/Sunday, Tuesday/Friday/Sunday). The dose can also start from 50 mg or 100 mg QOD at first (twice/three times weekly) and then has the option of switching to 150 mg or 200 mg (twice/three times weekly); or initially compound (I) is administered at high doses of 150 or 200 mg at first (twice/three times weekly) but is switched to 50 mg or 100 mg later (twice/three times weekly); wherein the dose change happens at any time during the treatment period.

1. A method of treating HBV infection in a human patient, comprising administering to said patient a pharmaceutical composition containing an active ingredient of compound (I) in an amount from 50 mg to 200 mg QOD or QW or Q2W with or without other anti-HBV drugs; wherein compound (I) is (1S)-1-[(2S,4R,5R)-5-(5-amino-2-oxo-thiazolo[4,5-d]pyrimidin-3-yl)-4-hydroxy-tetrahydrofuran-2-yl]propyl] acetate or pharmaceutically acceptable salt; wherein the entire treatment process comprises two or three Compound (I) treatment periods (“on-period”), and each adjacent two on-periods are separated by one “off-period” during which Compound (I) is not treated.

2. The method according to claim 1, wherein the entire treatment process starts with a preparation period; wherein the preparation period is 0-24 weeks, particularly 0 week, 4 weeks, 8 weeks, 12 weeks, 16 weeks, 20 weeks or 24 weeks.

3. The method according to claim 2, wherein the on-period is 6-24 weeks; off-period is 12-48 weeks; and the entire treatment process is 24-120 weeks, particularly 24 weeks, 36 weeks, 48 weeks, 60 weeks, 72 weeks, 84 weeks, 96 weeks, 108 weeks or 120 weeks.

4. The method according to claim 3, wherein compound (I) is administered at dose of 50 mg QOD, 100 mg QOD, 150 mg QOD, or 200 mg QOD without other anti-HBV drugs; wherein the entire treatment process is 48 weeks, preparation period is 0 week, the first on-period is 8-16 weeks, the first off-period is 16-32 weeks, the second on-period is 8-16 weeks.

5. The method according to claim 3, wherein compound (I) is administered at dose of 50 mg QOD, 100 mg QOD, 150 mg QOD, or 200 mg QOD without other anti-HBV drugs; wherein the entire treatment process is 36 weeks, preparation period is 0 week, the first on-period is 12 weeks, the first off-period is 12 weeks, the second on-period is 12 weeks.

6. The method according to claim 3, wherein compound (I) is administered at dose of 50 mg QOD, 100 mg QOD, 150 mg QOD, or 200 mg QOD with other anti-HBV drugs, the other anti-HBV drugs are administered to the patient on the first day of the entire treatment process.

7. The method according to claim 6, wherein the other anti-HBV drugs are one or two agents independently selected from anti-HBV nucleos(t)ide analogues, immune checkpoint inhibitor, immune activator, HBV CpAM, HBV capsid inhibitor, RIG-I agonist, Sting agonist, HBV therapeutic vaccine, HBV LNA, HBV entry inhibitor, cccDNA destabilizers, siRNA and HBsAg inhibitor.

8. The method according to claim 6, wherein the other anti-HBV drugs are one or two agents independently selected from ETV, TDF, TAF, lamivudine, telbivudine, clevudine, nivolumab, pembrolizumab, aterolizumab, avelumab, durvalumab, ipilimumab, tremelimumab, toripalimab, Sintilimab, camrelizumab, tislelizumab, ASC22, HLX10, CA-170, GLS4, QL-007, KL060332, compound (II), ABI-H0731, ABI-H2158, AB-506, JNJ-6379, JNJ-0440, Inarigivir, MK-1454, ADU-S100, ABX203, INO-1800, HB-110, TG1050, HepTcell, compound (III), Myrcludex B, AB-452, ARB-1467, ARO-HBV, AB-729, DCR-HBVS, Vir-2218, BB-103, Lunar-HBV, REP 2139 and REP 2165.

9. The method according to claim 7, wherein the other anti-HBV drug is HBV CpAM.

10. The method according to claim 9, wherein the HBV CpAM is compound (II), 34(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-15 2,2-dimethyl-propanoic acid, which is dosed at 200-1000 mg QD, particularly at 200 mg QD, 400 mg QD, 600 mg QD, 800 mg QD or 1000 mg QD.

11. The method according to claim 10, wherein the entire treatment process is 48 weeks, the preparation period is 12 weeks, the first on-period is 12 weeks, the first off-period is 12 weeks, the second on-period is 12 weeks.

12. The method according to claim 10, wherein the entire treatment process is 48 weeks, the preparation period is 0-8 weeks, the first on-period is 8-16 weeks, the first off-period is 12-24 weeks, the second on-period is 8-16 weeks.

13. The method according to claim 7, wherein the other anti-HBV drugs are HBV CpAM and NUC.

14. The method according to claim 13, wherein the HBV CpAM is compound (II) which is dosed at 200-1000 mg QD, particularly at 200 mg QD, 400 mg QD, 600 mg QD, 800 mg QD or 1000 mg QD; wherein NUC is ETV, TDF or TAF, which is dosed according to its country-specific label.

15. The method according to claim 14, wherein the entire treatment process is 48 weeks, the preparation period is 12 weeks, the first on-period is 12 weeks, the first off-period is 12 weeks, the second on-period is 12 weeks.

16. The method according to claim 14, wherein the entire treatment process is 48 weeks, the preparation period is 0-8 weeks, the first on-period is 8-16 weeks, the first off-period is 12-24 weeks, the second on-period is 8-16 weeks.

17. The method according to any one of claims 10-12 and 14-16, wherein compound (II) is administered at dose of 600 mg QD.

18. The method according to claim 7, wherein the other anti-HBV drug is immune checkpoint inhibitor.

19. The method according to claim 18, wherein the immune checkpoint inhibitor is antibody, macrocyclic peptide or small molecule targeting PD-1/PD-L1, CTLA4 or VISTA pathways.

20. The method according to claim 18 or 19, wherein the immune checkpoint inhibitor is selected from nivolumab, pembrolizumab, aterolizumab, avelumab, durvalumab, ipilimumab, tremelimumab, toripalimab, Sintilimab, camrelizumab, tislelizumab, ASC22, HLX10, and CA-170; particularly the immune check point inhibitor is nivolumab.

21. The method according to claim 20, wherein the entire treatment process is 48 weeks, the preparation period is 12 week, the first on-period is 12 weeks, the first off-period is 12 weeks, the second on-period is 12 weeks.

22. The method according to claim 20, wherein the entire treatment process is 36 weeks, the preparation period is 0 week, the first on-period is 12 weeks, the first off-period is 12 weeks, the second on-period is 12 weeks.

23. The method according to any one of claims 20-22, wherein the nivolumab is administered at dose of 0.3-5 mg/kg Q2W, Q3W or Q4W.

24. The method according to any one of claims 1-23, wherein compound (I) is administered at dose of 150 mg QOD.

25. The method according to any one of claims 1-23, wherein compound (I) is administered at dose of 100 mg QOD.

26. The method according to claim 1, wherein compound (I) is administered at high doses of 150 or 200 mg QOD, which is switched to low doses of 50 mg QOD or 100 mg QOD and then still has the option of switching to 50 mg QW or 100 QW, wherein the dose change happens at any time during the treatment period.

27. The method according to claim 1, wherein compound (I) is administered at low doses of 50 or 100 mg QW, which is switched to 50 mg QOD or 100 mg QOD and then still has the option of switching to 150 mg QOD or 200 mg QOD; or initially compound (I) is administered at low doses of 50 or 100 mg QOD but is switched to 150 mg QOD or 200 mg QOD; wherein the dose change happens at any time during the treatment period.

28. A method of treating COVID-19 or SARS-CoV-2 infection in a human patient or treating subject for SARS-CoV-2 or COVID-19 prophylaxis, comprising administering to said patient or subject a pharmaceutical composition containing an active ingredient of compound (I) in an amount from 50 mg to 200 mg QOD or QW or Q2W with or without one or more other anti-COVID-19 or anti-SARS-CoV-2 drugs; wherein compound (I) is (1S)-1-[(2S,4R,5R)-5-(5-amino-2-oxo-thiazolo[4,5-d]pyrimidin-3-yl)-4-hydroxy-tetrahydrofuran-2-yl]propyl] acetate or pharmaceutically acceptable salt; wherein Compound (I) is used in the treatment period of 1-12 weeks.

29. The method according to claim 28, wherein compound (I) is administrated at 50 mg QOD or QW or Q2W, 100 mg QOD or QW or Q2W, 150 mg QOD or QW or Q2W, or 200 mg QOD or QW or Q2W.

30. The method according to claim 28 or 29, wherein the anti-COVID-19 or anti-SARS-CoV-2 drug is independently selected from direct acting anti-SARS-CoV-2 agents, immune modulators and vaccine.

31. The method according to claims 28 to 30, wherein the anti-COVID-19 or anti-SARS-CoV-2 drug is independently selected from remdesivir, REGN-COV2, LY-CoV555, MK-4482/EIDD-2801, CD24Fc, T-COVID™, Itolizumab, AdCOVID™, BNT162b1/2, mRNA-1273, AZD1222/ChAdOx1, Ad5-vectored COVID-19 vaccine, CoronaVac, and NVX-CoV2373.

32. The method according to claim 28-31, wherein compound (I) is administered at high doses of 150 or 200 mg QOD, which is switched to low doses of 50 mg QOD or 100 mg QOD and then still has the option of switching to 50 mg QW or 100 QW, wherein the dose change happens at any time during the treatment period.

33. The method according to claim 28-32, wherein compound (I) is administered at low doses of 50 or 100 mg QW, which is switched to 50 mg QOD or 100 mg QOD and then still has the option of switching to 150 mg QOD or 200 mg QOD; or initially compound (I) is administered at low doses of 50 or 100 mg QOD but is switched to 150 mg QOD or 200 mg QOD; wherein the dose change happens at any time during the treatment period.

34. The method according to any one of claims 1-33, wherein compound (I) is administered at doses of 50 to 200 mg, twice weekly with dosing interval between 1-4 days, or three times weekly with dosing interval between 1-2 days; wherein the dose starts from 50 mg or 100 mg QOD at first, and then has the option of switching to 150 mg or 200 mg twice or three times weekly; or initially compound (I) is administered at high doses of 150 or 200 mg at first twice or three times weekly, but is switched to 50 mg or 100 mg later twice or three times weekly; wherein the dose change happens at any time during the treatment period.
